(19) United States
(12) Patent Application Publication
(10) Pub. No.: US 2006/0160863 A1
(43) Pub. Date: Jul. 20, 2006

(54) Polymorphic and amorphous forms of 2-{3-[(E)-2-(4,6-dimethyl-pyridin-2-yl)-vinyl]-1H-indazol-6-ylamino}-N-(4-hydroxy-but-2-ynyl)benzamide

(75) Inventors: Tim Edward Alcacio, San Diego, CA (US); Winnie Hoyan Chan, Placentia, CA (US); Mark Guzman, San Diego, CA (US); Asayuki Kamatani, San Diego, CA (US); Cynthia Palmer, San Diego, CA (US); Nabil Saeed, San Diego, CA (US); Jill Melissa Scott, San Diego, CA (US); Jayaram Kasturi Srirangam, San Diego, CA (US)

Correspondence Address: AGOURON PHARMACEUTICALS, INC., 10777 SCIENCE CENTER DRIVE, SAN DIEGO, CA, 92121, US

(73) Assignee: AGOURON PHARMACEUTICALS, INC.

(21) Appl. No.: 11/300,017

(22) Filed: Dec. 14, 2005

Related U.S. Application Data
(60) Provisional application No. 60/641,662, filed on Jan. 5, 2005.

(51) Int. Cl. A61K 31/4439; C07D 403/02

(52) U.S. Cl. 514/338; 546/275.7

(57) ABSTRACT

This invention provides several crystalline polymorphic forms, amorphous forms, and mixtures thereof of the compound of Formula I: 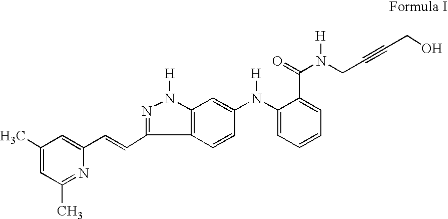 and pharmaceutical compositions made therewith, and to the therapeutic or prophylactic use of such compounds and pharmaceutical compositions, to treat ophthalmic diseases, such as age related macular degeneration and other diseases associated with unwanted ocular angiogenesis.

### CROSS REFERENCE TO RELATED APPLICATION

This application claims the benefit of U. S. Provisional Application Ser. No. 60/641,662 filed Jan. 5, 2005, the contents of which is hereby incorporated by reference in it's entirety.

### BACKGROUND OF THE INVENTION

U.S. patent application Ser. No. 10/737,655, which is incorporated herein by reference in its entirety, is directed to indazole compounds that modulate and/or inhibit the activity of certain protein kinases. Such compounds are useful for treating ophthalmic diseases such as age related macular degeneration (AMD) and diabetic retinopathy (DR), and other diseases associated with angiogenesis and/or cellular proliferation mediated by protein kinases.

One compound disclosed in the Ser. No. 10/737,655 application is 2-{3-[(E)-2-(4,6-dimethyl-pyridin-2-yl)-vinyl]-1H-indazol-6-ylamino}-N-(4-hydroxy-but-2-ynyl)-benzamide, the structure of which is shown below as Formula I:

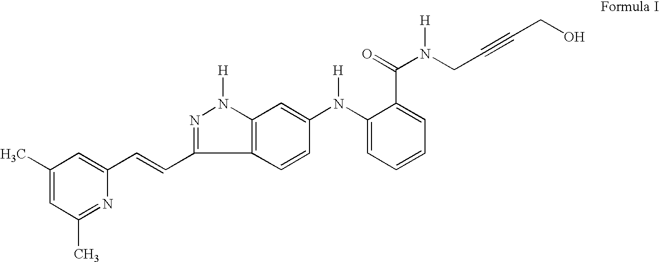

With respect to choroidal neovascularization (CNV), the compound of Formula I is known to be a potent inhibitor of vascular endothelial growth factor (VEGF) signaling—a known mechanism required of CNV.

To prepare pharmaceutical compositions containing the compound of Formula I for administration to mammals in accordance with the requirements of U.S. and international health registration authorities (e.g., FDA's Good Manufacturing Practices (“GMP”)), there is a need to produce the compound of Formula I in a stable form, such as a stable crystalline form having constant physical properties. Further, there is a need in the art to provide improved forms of the compound of Formula I having enhanced properties, such as improved solubility or oral bioavailability.

### SUMMARY OF THE INVENTION

The present invention provides crystalline forms, including polymorphic forms, amorphous forms, and solid forms, and mixtures thereof, of the compound of Formula I: 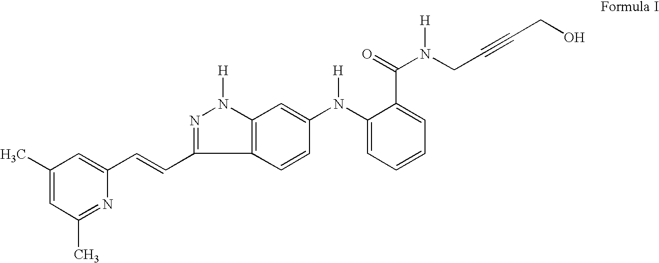
or pharmaceutically acceptable salts or solvates thereof; pharmaceutical compositions; and methods for treating disease conditions mediated by protein kinase activity, including ocular diseases such as age related macular degeneration, diabetic retinopathy, cell proliferation and angiogenesis; and methods for modulating the activity of protein kinase receptors, including VEGF receptors.

In one aspect, the invention provides crystalline forms of the compound of Formula I, or pharmaceutically acceptable salts or solvates thereof.

In another aspect, the invention provides crystalline forms of the compound of Formula I, wherein the crystalline form is a substantially pure polymorph of Form Ia.

In another aspect, the invention provides crystalline forms of the compound of Formula I, wherein the crystalline form has a powder X-ray diffraction pattern comprising peaks at diffraction angles (2θ) of about 24.4, 11.9 and 6.6.

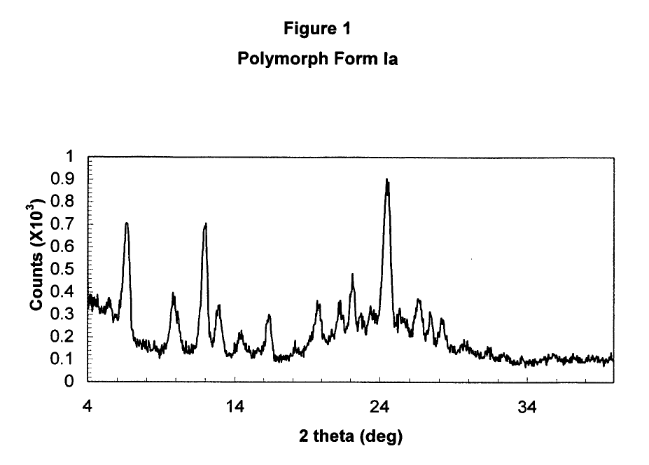

In another aspect, the invention provides crystalline forms of the compound of Formula I, wherein the crystalline form has a powder X-ray diffraction pattern comprising peaks at diffraction angles (2θ) essentially the same as shown in FIG. 1.

In another aspect, the invention provides crystalline forms of the compound of Formula I, wherein the crystalline form has a melting endotherm at about 122° C. as measured by a DSC when scanning at a scan rate of 10° C. per minute.

In another aspect, the invention provides crystalline forms of the compound of Formula I, wherein the crystalline form is a substantially pure polymorph of Form II.

In another aspect, the invention provides crystalline forms of the compound of Formula I, wherein the crystalline form has a powder X-ray diffraction pattern comprising peaks at diffraction angles (2θ) of about 27.3, 22.5 and 20.1.

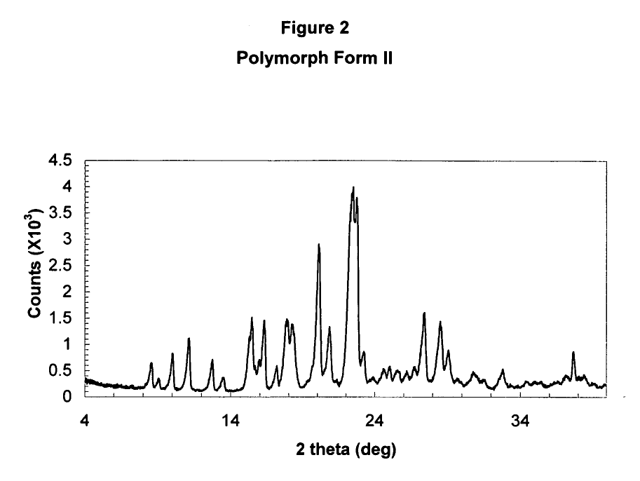

In another aspect, the invention provides crystalline forms of the compound of Formula I, wherein the crystalline form has a powder X-ray diffraction pattern comprising peaks at diffraction angles (2θ) essentially the same as shown in FIG. 2.

In another aspect, the invention provides crystalline forms of the compound of Formula I, wherein the crystalline form has a melting endotherm at about 208° C. as measured by a DSC when scanning at a scan rate of 10° C. per minute.

In another aspect, the invention provides crystalline forms of the compound of Formula I, wherein the crystalline form is a substantially pure polymorph of Form III.

In another aspect, the invention provides crystalline forms of the compound of Formula I, wherein the crystalline form has a powder X-ray diffraction pattern comprising peaks at diffraction angles (2θ) of about 32.1, 24.2 and 25.2.

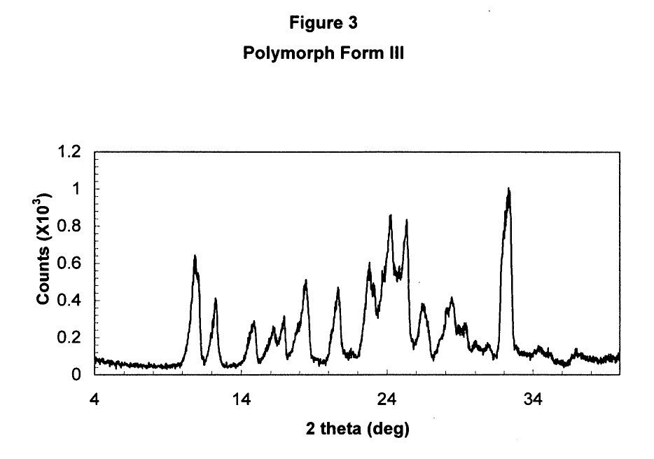

In another aspect, the invention provides crystalline forms of the compound of Formula I, wherein the crystalline form has a powder X-ray diffraction pattern comprising peaks at diffraction angles (2θ) essentially the same as shown in FIG. 3.

In another aspect, the invention provides crystalline forms of the compound of Formula I, wherein the crystalline form has a melting endotherm at about 179° C. as measured by a DSC when scanning at a scan rate of 10° C. per minute.

In another aspect, the invention provides crystalline forms of the compound of Formula I, wherein the crystalline form is a substantially pure polymorph of Form IV.

In another aspect, the invention provides crystalline forms of the compound of Formula I, wherein the polymorph has a powder X-ray diffraction pattern comprising peaks at diffraction angles (2θ) of about 24.7, 17.9 and 23.6.

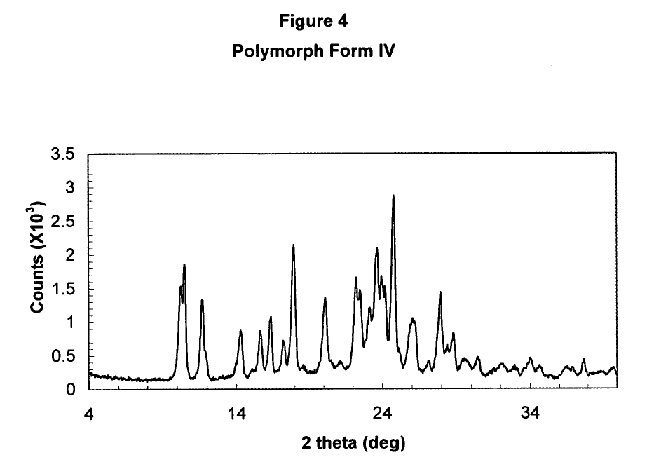

In another aspect, the invention provides crystalline forms of the compound of Formula I, wherein the crystalline form has a powder X-ray diffraction pattern comprising peaks at diffraction angles (2θ) essentially the same as shown in FIG. 4.

In another aspect, the invention provides crystalline forms of the compound of Formula I, wherein the crystalline form is characterized by an onset of crystal melting endotherm at about 205° C. when scanning at a scan rate of 10° C. per minute.

In another aspect, the invention provides crystalline forms of the compound of Formula I, wherein the crystalline form is a substantially pure polymorph of Form V.

In another aspect, the invention provides crystalline forms of the compound of Formula I, wherein the polymorph has a powder X-ray diffraction pattern comprising peaks at diffraction angles (2θ) of about 24.5, 23.4 and 25.4.

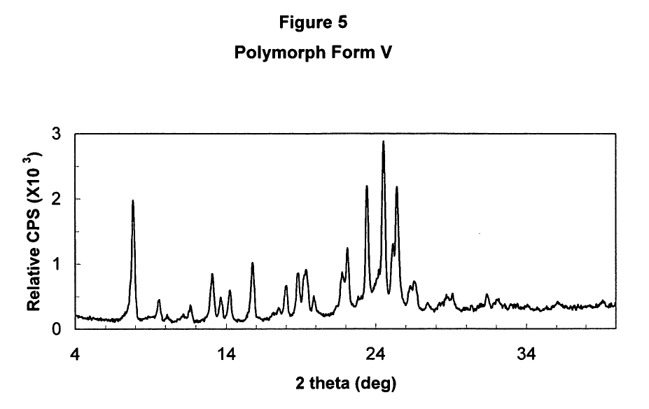

In another aspect, the invention provides crystalline forms of the compound of Formula I, wherein the crystalline form has a powder X-ray diffraction pattern comprising peaks at diffraction angles (2θ) essentially the same as shown in FIG. 5.

In another aspect, the invention provides crystalline forms of the compound of Formula I, wherein the crystalline form is characterized by an onset of crystal melting endotherm at about 133° C., 160° C. and 206° C. when scanning at a scan rate of 10° C. per minute.

In another aspect, the invention provides crystalline forms of the compound of Formula I, wherein the crystalline form is a substantially pure polymorph of Form VI.

In another aspect, the invention provides crystalline forms of the compound of Formula I, wherein the polymorph has a powder X-ray diffraction pattern comprising peaks at diffraction angles (2θ) of 24.8, 16.9 and 26.3.

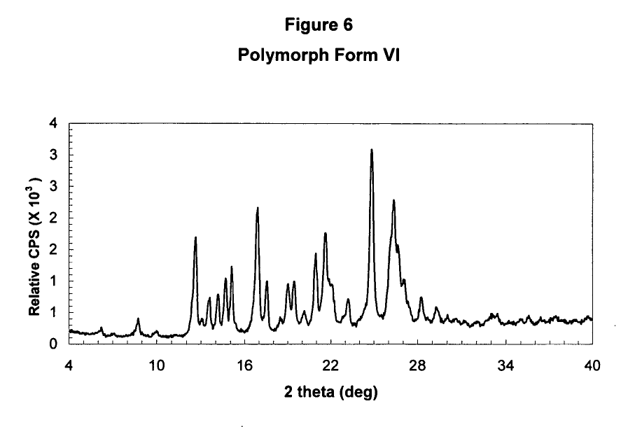

In another aspect, the invention provides crystalline forms of the compound of Formula I, wherein the crystalline form has a powder X-ray diffraction pattern comprising peaks at diffraction angles (2θ) essentially the same as shown in FIG. 6.

In another aspect, the invention provides crystalline forms of the compound of Formula I, wherein the crystalline form is characterized by an onset of crystal melting endotherm at about 133° C., 146° C and 203° C. when scanning at a scan rate of 10° C. per minute.

In another aspect, the invention provides amorphous forms of the compound of Formula I, or a pharmaceutically acceptable salt or solvate thereof.

In another aspect, the invention provides amorphous forms of the compound of Formula I, wherein the amorphous form has a powder X-ray diffraction pattern exhibiting a broad peak at diffraction angles (2θ) ranging from about 4 to about 40° without any of the sharp peaks characteristic of a crystalline form.

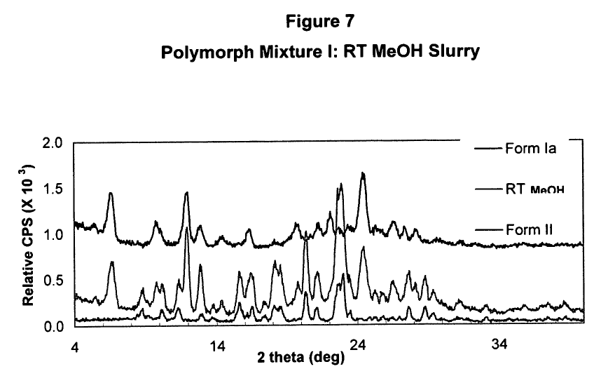

In another aspect, the invention provides amorphous forms of the compound of Formula I, wherein the amorphous form has a powder X-ray diffraction pattern essentially the same as shown in FIG. 7.

In another aspect, the invention provides solid forms of the compound of Formula I, or a pharmaceutically acceptable salt or solvate thereof, wherein the solid form is a mixture comprising at least two of the following solid forms: polymorph Forms IA, II, III, IV, and an amorphous form.

In another aspect, the invention provides solid forms of the compound of Formula I, wherein the solid form is a mixture of polymorph Form Ia and polymorph Form II.

In another aspect, the invention provides crystalline forms of the compound of Formula I, wherein the crystalline form is a substantially pure polymorph of Form Ia and Form II.

In another aspect, the invention provides crystalline forms of the compound of Formula I, wherein the polymorph has a powder X-ray diffraction pattern comprising peaks at diffraction angles (2θ) of about 11.8, 24.4 and 6.5.

In another aspect, the invention provides crystalline forms of the compound of Formula I, wherein the crystalline form has a powder X-ray diffraction pattern comprising peaks at diffraction angles (2θ) essentially the same as shown in FIG. 7.

In another aspect, the invention provides crystalline forms of the compound of Formula I, wherein the crystalline form is characterized by an onset of crystal melting endotherm at about 122° C. and about 206° C. when scanning at a scan rate of 10° C. per minute.

In another aspect, the invention provides pharmaceutical compositions comprising the crystalline forms of the compound of Formula I, wherein the crystalline form is a substantially pure polymorph of Form Ia.

In another aspect, the invention provides pharmaceutical compositions comprising the crystalline forms of the compound of Formula I, wherein the crystalline form is a substantially pure polymorph of Form II.

In another aspect, the invention provides pharmaceutical compositions comprising the crystalline forms of the compound of Formula I, wherein the crystalline form is a substantially pure polymorph of Form III.

In another aspect, the invention provides pharmaceutical compositions comprising the crystalline forms of the compound of Formula I, wherein the crystalline form is a substantially pure polymorph of Form IV.

In another aspect, the invention provides pharmaceutical compositions comprising the crystalline forms of the compound of Formula I, wherein the crystalline form is a substantially pure polymorph of Form V.

In another aspect, the invention provides pharmaceutical compositions comprising the crystalline forms of the compound of Formula I, wherein the crystalline form is a substantially pure polymorph of Form VI.

In another aspect, the invention provides pharmaceutical compositions comprising the amorphous forms of the compound of Formula I.

In another aspect, the invention provides pharmaceutical compositions comprising the solid forms of the compound of Formula I, wherein the solid form is a mixture comprising at least two of the following solid forms: polymorph Forms IA, II, III, IV, V, and an amorphous form.

In another aspect, the invention provides pharmaceutical compositions comprising the solid forms of the compound of Formula I, wherein the solid form is a mixture of polymorph Form la and polymorph Form II.

In another aspect, the invention provides pharmaceutical compositions comprising the crystalline forms of the compound of Formula I, wherein the crystalline form is a mixture of polymorph Form Ia and polymorph Form II.

In another aspect, the invention provides methods for treating mammalian disease conditions mediated by protein kinase activity, comprising administering to a mammal in need thereof a therapeutically effective amount of a pharmaceutical composition comprising the crystalline forms of the compound of Formula I (polymorph Forms IA, II, III, IV, V or VI); the amorphous forms of the compound of Formula I; or the solid forms of the compound of Formula I (polymorph Forms IA, II, III, IV, V or VI), or mixtures thereof.

In another aspect, the invention provides methods for treating mammalian disease conditions mediated by protein kinase activity, wherein the mammalian disease condition is associated with an ocular disease.

In another aspect, the invention provides methods for treating mammalian disease conditions mediated by protein kinase activity, wherein the mammalian disease condition is associated with age related macular degeneration, diabetic retinopathy, cell proliferation, or angiogenesis.

In another aspect, the invention provides methods for modulating the activity of protein kinase receptors, comprising contacting the kinase receptor with an effective amount of a crystalline form of the compound of Formula I (polymorph Forms IA, II, III, IV, V or VI); the amorphous forms of the compound of Formula I; or the solid forms of the compound of Formula I (polymorph Forms IA, II, III, IV, V or VI), or mixtures thereof.

In another aspect, the invention provides methods for modulating the activity of protein kinase receptors, wherein the protein kinase receptor is a VEGF receptor.

In another aspect, the invention provides processes for converting the substantially pure polymorph of Form IV to the substantially pure polymorph of Form II, comprising heating the substantially pure polymorph of Form IV to about 120° C.

In another aspect, the invention provides crystalline forms of the compound of Formula I, wherein the crystalline form is a mixture of polymorphs of Form II and Form IV.

In another aspect, the invention provides crystalline forms of the compound of Formula I, wherein the mixture of polymorphs is about 90% Form II and about 10% Form IV.

In another aspect, the invention provides crystalline forms of the compound of Formula I, wherein the mixture of polymorphs is about 80% Form II and about 20% Form IV.

In another aspect, the invention provides crystalline forms of the compound of Formula I, wherein the substantially pure mixture of polymorphs is about 70% Form II and about 30% Form IV.

In another aspect, the invention provides crystalline forms of the compound of Formula I, wherein the mixture of polymorphs is about 60% Form II and about 40% Form IV.

### BRIEF DESCRIPTION IF THE INVENTION

Having thus described the invention in general terms, reference will now be made to the accompanying drawings, wherein:

FIG. 1 is an X-ray powder diffraction diagram of polymorph Form Ia of the invention;

FIG. 2 is an X-ray powder diffraction diagram of polymorph Form II of the invention;

FIG. 3 is an X-ray powder diffraction diagram of polymorph Form III of the invention;

FIG. 4 is an X-ray powder diffraction diagram of polymorph Form IV of the invention;.

FIG. 5 is an X-ray powder diffraction diagram of polymorph Form V of the invention;

FIG. 6 is an X-ray powder diffraction diagram of polymorph Form VI of the invention;

FIG. 7 is an X-ray powder diffraction diagram of polymorph mixture I of the invention; and

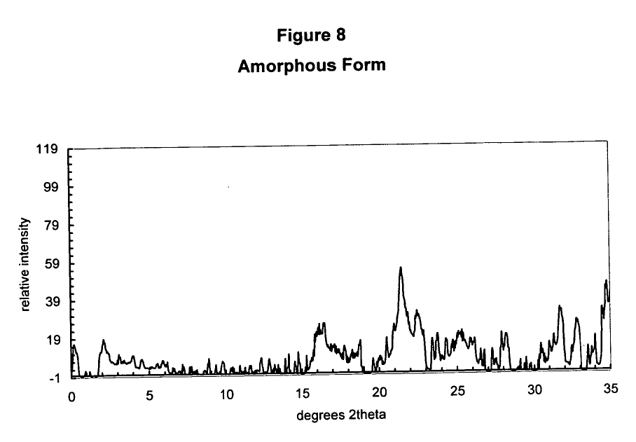

FIG. 8 is an X-ray powder diffraction diagram of an amorphous form of the invention.

### DETAILED DESCRIPTION OF THE INVENTION

Definitions

The following terms as used herein have the meanings indicated.

As used in the specification, and in the appended claims, the singular forms “a”, “an”, “the”, include plural referents unless the context clearly dictates otherwise.

The terms “comprising” and “including” are used in an open, non-limiting sense.

The term “polymorph” refers to a crystalline form of a compound with a distinct spatial lattice arrangement as compared to other crystalline forms of the same compound.

The term “amorphous” refers to a non-crystalline form of a compound.

The terms “A pharmaceutically acceptable salt” is intended to mean a salt that retains the biological effectiveness of the free acids and bases of the specified compound and that is not biologically or otherwise undesirable. A compound of the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, y-hydroxybutyrates, glycollates, tartrates, methane-sulfonates, propane-sulfonates, naphthalene-1-sulfonates, naphthalene-2-sulfonates, and mandelates.

Polymorphic and Amorphous Forms of the Compound of Formula I

The present invention provides several polymorph crystalline forms and an amorphous form of the compound of Formula I. Each crystalline or amorphous form of the compound can be characterized by one or more of the following: X-ray powder diffraction pattern (i.e., X-ray diffraction peaks at various diffraction angles (2θ), melting point onset (and onset of dehydration for hydrated forms) as illustrated by endotherms of a Differential Scanning Calorimetry (DSC) thermogram, aqueous solubility, light stability under International Conference on Harmonization (ICH) high intensity light conditions, and physical and chemical storage stability.

The X-ray powder diffraction pattern for each polymorph or amorphous form of the invention was measured on a Shimadzu XRD-6000 X-ray diffractometer equipped with a Cu X-ray source operated at 40 kV and 50 mA. Samples were placed in a sample holder and then packed and smoothed with a glass slide. During analysis, the samples were rotated at 60 rpm and analyzed from angles of 4 to 40° (θ-2θ) at 5°/min with a 0.04° step or at 2°/min with a 0.02 ° step. If limited material was available, samples were placed on a silicon plate (zero background) and analyzed without rotation. One of skill in the art will appreciate that the peak positions (2θ) will show some inter-apparatus variability, typically as much as +0.1° or −0.1°. Accordingly, where the solid forms of the present invention are described as having a powder X-ray diffraction pattern essentially the same as that shown in a given figure, the term “essentially the same” is intended to encompass such inter-apparatus variability in diffraction peak positions.

The DSC thermographs were obtained using a Mettler Toledo DSC821^{e} instrument at a scan rate of 10° C. per minute over a temperature range of 30 to 250° C. Samples were weighed into 40 μl aluminum crucibles that were sealed and punctured with a single hole. The extrapolated onset of melting temperature and, where applicable, the onset of dehydration temperature, were calculated.

Depending on several factors, the endotherms exhibited by the compounds of the invention may vary (by about 0.01-5° C. for crystal polymorph melting and by about 0.01-20° C. for polymorph dehydration) above or below the endotherms depicted in the appended figures. Factors responsible for such variance include the rate of heating (i.e., the scan rate) at which the DSC analysis is conducted, the way the DSC onset temperature is defined and determined, the calibration standard used, instrument calibration, the relative humidity and the chemical purity of the sample. For any given sample, the observed endotherms may also differ from instrument to instrument; however, it will generally be within the ranges defined herein provided the instruments are calibrated similarly.

The polymorph or amorphous forms of the invention are preferably substantially pure, meaning each polymorph or amorphous form of the compound of Formula I includes less than 10%, preferably less than 5%, preferably less than 3%, preferably less than 1% by weight of impurities, including other polymorph or amorphous forms of the compound.

The solid forms of the present invention may also exist together in a mixture. Mixtures of polymorphs and/or the amorphous form of the present invention will have X-ray diffraction peaks characteristic of each of the polymorphs and/or amorphous forms present in the mixture. For example, a mixture of two polymorphs will have a powder X-ray diffraction pattern that is a convolution of the X-ray diffraction patterns corresponding to the substantially pure polymorphs.

Polymorph Form Ia

Polymorph Form Ia is a hydrated crystal of the compound of Formula I. DSC of Form Ia shows a melting endotherm at 122° C. when scanning at a scan rate of 10° C. per minute. The aqueous solubility of Form Ia is 0.8 μg/mL in 0.1 M sodium phosphate buffer (pH 7.4). The X-ray powder diffraction data of Form Ia is as follows:

| 2(Θdeg) | I/I₁₀₀(%) |
|---|---|
| 5.4 | 8 |
| 6.6 | 67 |
| 9.9 | 29 |
| 11.9 | 76 |
| 12.9 | 27 |
| 14.4 | 12 |
| 15.5 | 3 |
| 16.3 | 25 |
| 18.2 | 4 |
| 19.0 | 5 |
| 19.7 | 28 |
| 20.6 | 7 |
| 21.3 | 24 |
| 22.1 | 37 |
| 22.7 | 17 |
| 23.4 | 20 |
| 24.4 | 100 |
| 25.3 | 18 |
| 26.6 | 28 |
| 27.4 | 18 |
| 28.2 | 17 |
| 29.8 | 5 |
| 31.3 | 4 |
| 35.8 | 4 |

Polymorph Form II

Polymorph Form II is an anhydrous crystal of the compound of Formula I. DSC of Form II shows a melting endotherm at 208° C. when scanning at a scan rate of 10° C. per minute. The aqueous solubility of Form II is 1.3 μg/ml in 0.1 M sodium phosphate buffer (pH 7.4). The X-ray powder diffraction data of Form II is as follows:

| 2Θ(deg) | I/I₁₀₀(%) |
|---|---|
| 8.5 | 8 |
| 9.0 | 3 |
| 10.0 | 11 |
| 11.1 | 18 |
| 12.7 | 10 |
| 13.5 | 5 |
| 15.4 | 30 |
| 16.2 | 25 |
| 17.1 | 7 |
| 18.0 | 32 |
| 20.1 | 56 |
| 20.8 | 23 |
| 22.5 | 100 |
| 23.2 | 11 |
| 24.8 | 6 |
| 25.5 | 6 |
| 26.2 | 4 |
| 26.7 | 6 |
| 27.3 | 29 |
| 28.5 | 27 |
| 29.1 | 14 |
| 30.9 | 7 |
| 31.5 | 4 |
| 32.7 | 8 |
| 37.1 | 5 |
| 38.3 | 5 |

Polymorph Form III

Polymorph Form III is a hydrated crystal of the compound of Formula I. DSC of Form III shows a melting endotherm at 179° C. when scanning at a scan rate of 10° C. per minute. The aqueous solubility of Form III is 3.2 μg/ ml in 0.1 M sodium phosphate buffer (pH 7.4). The X-ray powder diffraction data of Form III is as follows:

| 2Θ(deg) | I/I₁₀₀(%) |
|---|---|
| 0.9 | 63 |
| 12.2 | 33 |
| 14.7 | 24 |
| 16.1 | 18 |
| 16.8 | 23 |
| 17.9 | 20 |
| 18.3 | 45 |
| 20.5 | 38 |
| 22.8 | 48 |
| 24.2 | 74 |
| 25.2 | 72 |
| 26.5 | 26 |
| 28.3 | 31 |
| 29.2 | 16 |
| 30.0 | 7 |
| 30.9 | 7 |
| 32.1 | 100 |
| 33.1 | 4 |
| 33.5 | 4 |
| 34.4 | 7 |
| 35.1 | 5 |
| 37.1 | 5 |

Polymorph Form IV

Polymorph Form IV is an anhydrous crystal of the compound of Formula I. DSC of Form IV shows a melting endotherm at 205° C. when scanning at a scan rate of 10° C. per minute. The aqueous solubility of Form IV is 1.3 μg/ml in 0.1 M sodium phosphate buffer (pH 7.4). The X-ray powder diffraction data of Form IV is as follows:

| 2Θ(deg) | I/I₁₀₀(%) |
|---|---|
| 10.2 | 55 |
| 10.5 | 68 |
| 11.7 | 47 |
| 14.2 | 27 |
| 15.1 | 3 |
| 15.6 | 26 |
| 16.3 | 33 |
| 17.2 | 19 |
| 17.9 | 77 |
| 18.6 | 4 |
| 20.1 | 45 |
| 20.5 | 5 |
| 21.1 | 4 |
| 22.2 | 52 |
| 22.5 | 46 |
| 23.1 | 34 |
| 23.6 | 71 |
| 24.0 | 52 |
| 24.7 | 100 |
| 25.2 | 7 |
| 26.0 | 29 |
| 27.1 | 5 |
| 27.9 | 46 |
| 28.4 | 15 |
| 28.8 | 23 |
| 29.6 | 8 |
| 30.4 | 11 |
| 31.5 | 3 |
| 32.1 | 7 |
| 32.9 | 6 |
| 33.5 | 4 |
| 34.0 | 11 |
| 34.6 | 7 |
| 36.5 | 6 |
| 36.9 | 6 |
| 37.7 | 11 |
| 39.7 | 5 |

Polymorph Form V

Polymorph Form V is an ethanol solvated crystal of the compound of Formula I. DSC of Form V shows melting endotherms at 133° C., 160° C. and 206° C. when scanning at a scan rate of 10° C. per minute. The X-ray powder diffraction data of Form IV is as follows:

| 2Θ(deg) | I/I₁₀₀(%) |
|---|---|
| 7.1 | 3 |
| 7.8 | 72 |
| 9.5 | 13 |
| 10.1 | 3 |
| 11.1 | 4 |
| 11.6 | 9 |
| 13.0 | 27 |
| 13.6 | 13 |
| 14.2 | 18 |
| 15.7 | 34 |
| 17.0 | 3 |
| 17.5 | 6 |
| 17.9 | 19 |
| 18.8 | 27 |
| 19.3 | 28 |
| 19.8 | 11 |
| 21.3 | 3 |
| 21.7 | 22 |
| 22.0 | 36 |
| 22.8 | 5 |
| 23.4 | 74 |
| 23.8 | 11 |
| 24.1 | 18 |
| 24.5 | 100 |
| 25.1 | 37 |
| 25.4 | 73 |
| 26.2 | 13 |
| 26.5 | 17 |
| 27.4 | 4 |
| 28.2 | 4 |
| 28.6 | 9 |
| 29.0 | 10 |
| 30.9 | 4 |
| 31.3 | 9 |
| 31.8 | 4 |
| 32.0 | 6 |
| 32.9 | 3 |
| 33.2 | 3 |
| 36.0 | 5 |
| 39.0 | 3 |

Polymorph Form VI

Polymorph Form VI is an ethyl acetate solvate of the compound of Formula I. DSC of Form VI shows melting endotherms at 133° C., 146° C. and 203° C. when scanning at a scan rate of 10° C. per minute. The X-ray powder diffraction data of Form IV is as follows:

| 2Θ(deg) | I/I₁₀₀(%) |
|---|---|
| 6.2 | 4 |
| 8.7 | 9 |
| 12.6 | 54 |
| 13.0 | 7 |
| 13.5 | 19 |
| 14.1 | 20 |
| 14.7 | 28 |
| 15.1 | 32 |
| 16.5 | 8 |
| 16.9 | 70 |
| 17.5 | 27 |
| 18.5 | 6 |
| 19.0 | 24 |
| 19.4 | 26 |
| 20.1 | 7 |
| 20.9 | 39 |
| 21.6 | 52 |
| 22.1 | 20 |
| 22.8 | 3 |
| 23.2 | 14 |
| 24.3 | 7 |
| 24.8 | 100 |
| 26.0 | 26 |
| 26.3 | 68 |
| 26.7 | 41 |
| 27.0 | 24 |
| 27.5 | 6 |
| 28.2 | 14 |
| 29.3 | 8 |
| 30.0 | 3 |
| 33.0 | 5 |
| 33.4 | 6 |
| 35.5 | 5 |
| 37.3 | 4 |

Amorphous Forms

The amorphous form of the compound of Formula I has an X-ray powder diffraction which shows typical amorphous broad hump-peaks from 4 to 40°, without any sharp peaks characteristic of crystalline form.

Polymorph Mixture I

The crystalline and amorphous forms discussed above may also exist in mixtures, wherein the solid form exists as a mixture comprising at least two of the solid forms discussed above. Polymorph mixtures containing Form Ia and Form II are characterized by a DSC melting endotherms at 122° C. and 206° C. (similar to Form Ia and Form II, respectively), when scanning at a scan rate of 10° C. per minute. The X-ray powder diffraction data of polymorph mixture of Form Ia and Form II is as follows:

| 2Θ(deg) | I/I₁₀₀(%) |
|---|---|
| 5.3 | 12 |
| 6.5 | 71 |
| 9.2 | 7 |
| 9.7 | 52 |
| 10.2 | 9 |
| 11.3 | 4 |
| 11.8 | 100 |
| 12.8 | 39 |
| 14.2 | 25 |
| 14.7 | 3 |
| 15.6 | 9 |
| 16.2 | 34 |
| 18.1 | 10 |
| 19.2 | 12 |
| 19.6 | 45 |
| 20.8 | 10 |
| 21.3 | 31 |
| 22.1 | 47 |
| 22.5 | 22 |
| 23.5 | 23 |
| 24.4 | 98 |
| 25.2 | 13 |
| 25.8 | 12 |
| 26.2 | 14 |
| 26.6 | 26 |
| 27.3 | 17 |
| 28.2 | 24 |
| 28.7 | 4 |
| 29.6 | 9 |
| 30.2 | 5 |
| 30.5 | 3 |
| 31.2 | 11 |
| 32.3 | 4 |
| 33.3 | 3 |
| 33.5 | 3 |
| 35.6 | 7 |
| 37.2 | 3 |
| 38.4 | 3 |
| 38.8 | 3 |
| 39.6 | 5 |

Pharmaceutical Compiositions

The active agents (i.e., the polymorph or amorphous forms, or mixtures thereof, of the compound of Formula I described herein) of the invention may be formulated into pharmaceutical compositions suitable for both veterinary and human medical use. Pharmaceutical compositions of the invention comprise a therapeutically effective amount of the active agent and one or more inert, pharmaceutically acceptable carriers, and optionally any other therapeutic ingredients, stabilizers, or the like. The carrier(s) must be pharmaceutically acceptable in the sense of being compatible with the other ingredients of the formulation and not unduly deleterious to the recipient thereof. The compositions may further include diluents, buffers, binders, disintegrants, thickeners, lubricants, preservatives (including antioxidants), flavoring agents, taste-masking agents, inorganic salts (e.g., sodium chloride), antimicrobial agents (e.g., benzalkonium chloride), sweeteners, antistatic agents, surfactants (e.g., polysorbates such as “TWEEN 20” and “TWEEN 80”, and pluronics such as F68 and F88, available from BASF), sorbitan esters, lipids (e.g., 15 phospholipids such as lecithin and other phosphatidylcholines, phosphatidylethanolamines, fatty acids and fatty esters, steroids (e.g., cholesterol)), and chelating agents (e.g., EDTA, zinc and other such suitable cations). Other pharmaceutical excipients and/or additives suitable for use in the compositions according to the invention are listed in “Remington: The Science & Practice of Pharmacy”, 19^{th} ed., Williams & Williams, (1995), and in the “Physician's Desk Reference”, 52^{nd} ed., Medical Economics, Montvale, N.J. (1998), and in “Handbook of Pharmaceutical Excipients”, Third Ed., Ed. A. H. Kibbe, Pharmaceutical Press, 2000. The active agents of the invention may be formulated in compositions including those suitable for oral, rectal, topical, nasal, ophthalmic, or parenteral (including intraperitoneal, intravenous, subcutaneous, or intramuscular injection) administration.

The amount of the active agent in the formulation will vary depending upon a variety of factors, including dosage form, the condition to be treated, target patient population, and other considerations, and will generally be readily determined by one skilled in the art. A therapeutically effective amount will be an amount necessary to modulate, regulate, or inhibit a protein kinase. In practice, this will vary widely depending upon the particular active agent, the severity of the condition to be treated, the patient population, the stability of the formulation, and the like. Compositions will generally contain anywhere from about 0.001% by weight to about 99% by weight active agent, preferably from about 0.01% to about 5% by weight active agent, and more preferably from about 0.01% to 2% by weight active agent, and will also depend upon the relative amounts of excipients/additives contained in the composition.

A pharmaceutical composition of the invention is administered in conventional dosage form prepared by combining a therapeutically effective amount of an active agent as an active ingredient with one or more appropriate pharmaceutical carriers according to conventional procedures. These procedures may involve mixing, granulating and compressing or dissolving the ingredients as appropriate to the desired preparation.

The pharmaceutical carrier employed may be either a solid or liquid. Exemplary solid carriers include lactose, sucrose, talc, gelatin, agar, pectin, acacia, magnesium stearate, stearic acid and the like. Exemplary liquid carriers include syrup, peanut oil, olive oil, water and the like. Similarly, the carrier may include time-delay or time-release materials known in the art, such as glyceryl monostearate or glyceryl distearate alone or with a wax, ethylcellulose, hydroxypropylmethylcellulose, methylmethacrylate and the like.

A variety of pharmaceutical forms can be employed. Thus, if a solid carrier is used, the preparation can be tableted, placed in a hard gelatin capsule in powder or pellet form or in the form of a troche or lozenge. The amount of solid carrier may vary, but generally will be from about 25 mg to about 1 g. If a liquid carrier is used, the preparation can be in the form of syrup, emulsion, soft gelatin capsule, sterile injectable solution or suspension in an ampoule or vial or non-aqueous liquid suspension.

To obtain a stable water-soluble dose form, a pharmaceutically acceptable salt of an active agent is dissolved in an aqueous solution of an organic or inorganic acid, such as 0.3M solution of succinic acid or citric acid. If a soluble salt form is not available, the active agent may be dissolved in a suitable cosolvent or combinations of cosolvents. Examples of suitable cosolvents include, but are not limited to, alcohol, propylene glycol, polyethylene glycol 300, polysorbate 80, gylcerin and the like in concentrations ranging from 0-60% of the total volume. The composition may also be in the form of a solution of a salt form of the active agent in an appropriate aqueous vehicle such as water or isotonic saline or dextrose solution.

It will be appreciated that the actual dosages of the active agents used in the compositions of this invention will vary according to the particular complex being used, the particular composition formulated, the mode of administration and the particular site, host and disease being treated. Those skilled in the art using conventional dosage-determination tests in view of the experimental data for an agent can ascertain optimal dosages for a given set of conditions. For oral administration, an exemplary daily dose generally employed is from about 0.001 to about 1000 mg/kg of body weight, more preferably from about 0.001 to about 50 mg/kg body weight, with courses of treatment repeated at appropriate intervals. Administration of prodrugs is typically dosed at weight levels that are chemically equivalent to the weight levels of the fully active form.

The compositions of the invention may be manufactured in manners generally known for preparing pharmaceutical compositions, e.g., using conventional techniques such as mixing, dissolving, granulating, dragee-making, levigating, emulsifying, encapsulating, entrapping or lyophilizing. Pharmaceutical compositions may be formulated in a conventional manner using one or more physiologically acceptable carriers, which may be selected from excipients and auxiliaries that facilitate processing of the active compounds into preparations that can be used pharmaceutically.

Proper formulation is dependent upon the route of administration chosen. For injection, the agents of the invention may be formulated into aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration intranasally or by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator and the like may be formulated containing a powder mix of the compound and a suitable powder base such as lactose or starch.

The compounds may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

For administration to the eye, the active agent is delivered in a pharmaceutically acceptable ophthalmic vehicle such that the compound is maintained in contact with the ocular surface for a sufficient time period to allow the compound to penetrate the corneal and internal regions of the eye, including, for example, the anterior chamber, posterior chamber, vitreous body, aqueous humor, vitreous humor, cornea, iris/cilary, lens, choroid/retina and selera. The pharmaceutically acceptable ophthalmic vehicle may be, for example, an ointment, vegetable oil, or an encapsulating material. A compound of the invention may also be injected directly into the vitreous and aqueous humor or subtenon.

Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides.

In addition to the formulations described above, the compounds may also be formulated as a depot preparation. Such long-acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt.

A pharmaceutical carrier for hydrophobic compounds is a cosolvent system comprising benzyl alcohol, a nonpolar surfactant, a water-miscible organic polymer, and an aqueous phase. The cosolvent system may be a VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) contains VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid- or gel-phase carriers or excipients. Examples of such carriers or excipients include calcium carbonate, calcium phosphate, sugars, starches, cellulose derivatives, gelatin, and polymers such as polyethylene glycols.

Methods

The inventive compounds and compositions are useful for mediating the activity of protein kinases. More particularly, the compounds are useful as anti-angiogenesis agents and as agents for modulating and/or inhibiting the activity of protein kinases, such as the activity associated with VEGF, FGF, CDK complexes, TEK, CHK1, LCK, FAK, and phosphorylase kinase among others, thus providing treatments for cancer or other diseases associated with cellular proliferation mediated by protein kinases in mammals, including humans.

Therapeutically effective amounts of the agents of the invention may be administered, typically in the form of a pharmaceutical composition, to treat diseases mediated by modulation or regulation of protein kinases. An “effective amount” is intended to mean that amount of an agent that, when administered to a mammal in need of such treatment, is sufficient to effect treatment for a disease mediated by the activity of one or more protein kinases, such as tyrosine kinases. Thus, a therapeutically effective amount of a compound of the invention is a quantity sufficient to modulate, regulate, or inhibit the activity of one or more protein kinases such that a disease condition that is mediated by that activity is reduced or alleviated. The effective amount of a given compound will vary depending upon factors such as the disease condition and its severity and the identity and condition (e.g., weight) of the mammal in need of treatment, but can nevertheless be routinely determined by one skilled in the art. “Treating” is intended to mean at least the mitigation of a disease condition in a mammal, such as a human, that is affected, at least in part, by the activity of one or more protein kinases, such as tyrosine kinases, and includes: preventing the disease condition from occurring in a mammal, particularly when the mammal is found to be predisposed to having the disease condition but has not yet been diagnosed as having it; modulating and/or inhibiting the disease condition; and/or alleviating the disease condition. Exemplary disease conditions include diabetic retinopathy, neovascular glaucoma, rheumatoid arthritis, psoriasis, age-related macular degeneration (AMD), and cancer (solid tumors).

The activity of the inventive compounds as modulators of protein kinase activity, such as the activity of kinases, may be measured by any of the methods available to those skilled in the art, including in vivo and/or in vitro assays. Examples of suitable assays for activity measurements include those described in Parast C. et al., BioChemistry, 37, 16788-16801 (1998); Jeffrey et al., Nature, 376, 313-320 (1995); WIPO International Publication No. WO 97/34876; and WIPO International Publication No. WO 96/14843.

### EXAMPLES

The following examples are given to illustrate the invention, but should not be considered as limitations of the invention. Unless otherwise indicated, all temperatures are set forth in degrees Celsius and all parts and percentages are by weight. HPLC data was obtained using a Hewlett Packard HP-1100 HPLC.

### Example 1

### Polymorph Form Ia

The compound of Formula I (100 g, 92% pure by HPLC) is added to a solution of 20% methanol in methylene chloride (5 L). The mixture is concentrated under reduced pressure to approximately 80% of the original volume. The solids are filtered and dried under reduced pressure to provide 95 g of Form Ia.

### Example 2

### Polymorph Form II

The compound of Formula I (100 g) is added to a 1:1 solution of methyl t-butyl ether and ethyl acetate (5 L). The mixture is concentrated under reduced pressure and the residue is added to methanol (5 L). The methanol solution is heated to reflux for 12 hours, cooled to room temperature, and the solids are filtered and dried under reduced pressure to provide 95 g of Form

### Example 3

### Polymorph Form III

The compound of Formula I (100 g) is added to a solution of tetrahydrofuran (2.5 L) and ethyl acetate (2.5 L) and the mixture is heated to reflux for 12 hours. The formation of an oily precipitate is observed, which transforms into a solid upon cooling the solution overnight to room temperature. The solids are filtered and dried under reduced pressure to provide 95 g of Form III.

### Example 4

### Polymorph Form IV

The compound of Formula I (100 g) is added to a solution of tetrahydrofuran (2.5 L) and ethyl acetate (2.5 L) and the mixture is heated to reflux for 12 hours. The formation of an oily precipitate is observed which transforms into a solid upon cooling the solution overnight to room temperature. The mixture is filtered and dried under reduced pressure to provide 95 g of Form IV.

### Example 5

### Polymorph Form V

The compound of Formula I (1009) is added to ethanol (7 L) and the mixture is heated to reflux for 1 hour. The mixture is concentrated under reduced pressure to approximately 4 L. Several Form II crystals are added to the concentrate, and the mixture is concentrated under reduced pressure to approximately 500 mL. The solids are filtered and washed with ice-cold ethanol. The solids are dried under reduced pressure to provide 95 g of Form V (a 1:1 ethanol solvate).

### Example 6

### Polymorph Form VI

The compound of Formula I (25 g) is dissolved in tetrahydrofuran (250 mL) by warming the mixture to 45° C. The solution is evaporated under reduced pressure (˜60 mbar) with a constant feed of ethyl acetate (500 mL total) in such a rate to maintain approximately 250 mL total volume. The resulting brown suspension is cooled to room temperature followed by further cooling with an ice bath. The precipitated brown solids (amorphous) are filtered and washed with cold ethyl acetate (100 mL). On standing, spontaneous crystallization occurred, and the crystallized solids are filtered, washed with cold ethyl acetate (100 mL) and dried in a vacuum oven (70° C. and 25 mm Hg) to provide Form VI (a 1:1 ethyl acetate solvate).

### Example 7

### Amorphous Forms

These experiments are performed on a Crystallics in a hasteloy C22 stainless steel 316 L well plate. The plates contain 96 individual wells with a 50 μL volume. A stock solution was made by dissolving 3.5 g of Form IV in 400 mL of tetrahydrofuran. The stock is added to a 96 well plate to produce a product having concentration of 8.3, and 16.6 g/100 mL Heptanes. The solution is heated at a rate of 4.8° C. per min to 80° C. and held at 80° C. for 1 hour. The solution is cooled at a rate of 0.6° C. per minute to 20° C. and held at 20° C. for 1 hour. The solids are separated via filtration and dried under reduced pressure to provide an amorphous form.

### Example 8

### Polymorph Mixture I

The crystalline and amorphous forms discussed above may also exist in mixtures, wherein the solid form exists as a mixture comprising at least two of the solid forms discussed above.

For example, polymorph mixtures containing Form Ia and Form II are prepared by adding the compound of Formula I to methanol (15 mg/2mL solvent), and stirring the mixture at room temperature for 24 hours. The resulting slurry is filtered and evaporated under reduced pressure to provide polymorph mixtures containing Form Ia and Form II.

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A crystalline form of the compound of Formula I: 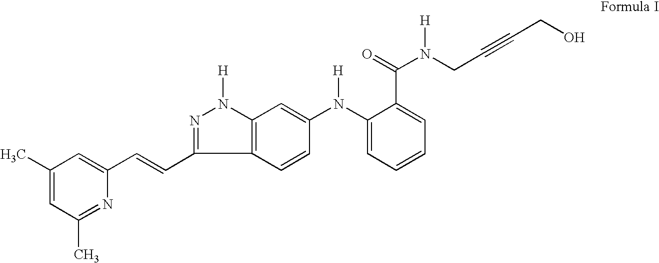
or a pharmaceutically acceptable salt or solvate thereof.

2. The crystalline form of claim 1, wherein the crystalline form is a substantially pure polymorph of any of Forms Ia, II, III, IV, V or VI.

3. The crystalline form of claim 1, wherein the crystalline form is a substantially pure polymorph of Form II.

4. The crystalline form of claim 3, wherein the crystalline form has a powder X-ray diffraction pattern comprising peaks at diffraction angles (2θ) of about 27.3, 22.5 and 20.1.

5. The crystalline form of claim 3, wherein the crystalline form has a powder X-ray diffraction pattern comprising peaks at diffraction angles (2θ) essentially the same as shown in FIG. 2.

6. The crystalline form of claim 3, wherein the crystalline form has a melting endotherm at about 208° C. as measured by a DSC when scanning at a scan rate of 10° C. per minute.

7. The crystalline form of claim 1, wherein the crystalline form is a substantially pure polymorph of Form Ia and Form II or is a substantially pure polymorph of Form II and Form IV.

8. An amorphous form of the compound of Formula I: 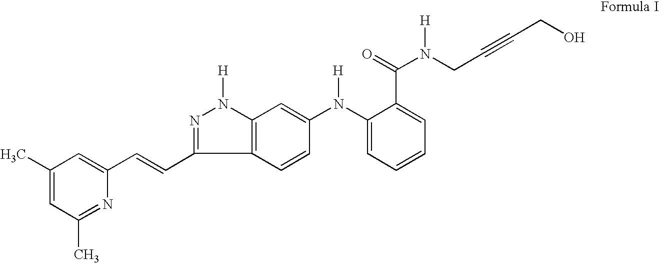
or a pharmaceutically acceptable salt or solvate thereof.

9. A solid form of the compound of Formula I: 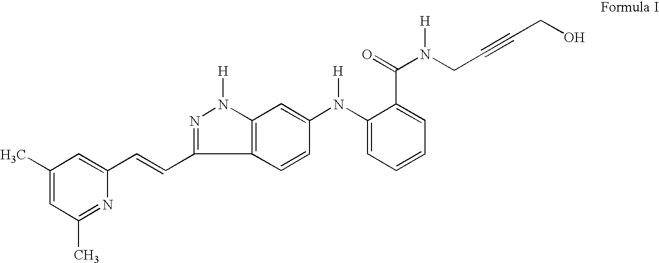
or a pharmaceutically acceptable salt or solvate thereof, wherein the solid form is a mixture comprising at least two of the following solid forms: polymorph Form Ia, II, III, IV and an amorphous form.

10. A pharmaceutical composition comprising any of the crystalline, amorphous or solid forms of claims 1 to 9.

11. A method of treating a mammalian disease condition mediated by protein kinase activity, comprising administering to a mammal in need thereof a therapeutically effective amount of the pharmaceutical composition of claim 10.

12. A method according to claim 11, wherein the mammalian disease condition is associated with age related macular degeneration, diabetic retinopathy, cell proliferation or angiogenesis.

13. A method of modulating the activity of a protein kinase receptor, comprising contacting the kinase receptor with an effective amount of a crystalline, amorphous or solid form according to claims 1 to 9.

14. The method according to claim 13, wherein the protein kinase receptor is a VEGF receptor.

15. A process for converting the substantially pure polymorph of Form IV of claim 2 to the substantially pure polymorph of Form II of claim 3, comprising heating the substantially pure polymorph of Form IV to about 120° C.
